(19) United States
(12) Patent Application Publication
(10) Pub. No.: US 2006/0095077 A1
(43) Pub. Date: May 4, 2006

(54) Expandable fixation structures

(76) Inventor: Carole A. Tronnes, Stillwater, MN (US)

Correspondence Address: SHUMAKER & SIEFFERT, P. A., 8425 SEASONS PARKWAY, SUITE 105, ST. PAUL, MN, 55125, US

(21) Appl. No.: 10/977,330

(22) Filed: Oct. 29, 2004

(51) Int. Cl. A61N 1/00; A61N 1/02

(52) U.S. Cl. 607/2

(57) ABSTRACT

In general, the invention is directed to a medical device implantable in a body of a patient. The medical device includes a non-expandable element, constructed of a biocompatible material such as silicone or polyurethane. The device also includes one or more expandable elements constructed of a hydrogel material. During implantation, the expandable elements are in a small, dehydrated state. When implanted in the body of a patient, the expandable elements absorb water from the body tissues and assume a larger, hydrated state, and resist migration of the implanted device.

### FIELD OF THE INVENTION

The invention relates to implantable medical devices implantable in a human or animal body and, more particularly, fixation structures for securing implantable medical devices.

### BACKGROUND

Many implantable medical devices include components that are deployed in particular areas within a human or animal body. In one example, a neurostimulator deployed proximate to targeted tissue includes electrodes that deliver an electrical stimulation therapy to the tissue. In another example, an electrical sensor deployed proximate to a muscle senses activation of the muscle. With these and other implantable devices, it can be desirable that one or more components remain substantially anchored, so that the components will be less likely to migrate from the desired site of sensing or therapy.

Devices that restrict migration of an implanted medical device or a component thereof are called “fixation structures.” Fixation structures can anchor a medical device to an anatomical feature, such as an organ or a bone. Fixation structures do not necessarily restrict all motion of the implanted device or its component, but generally reduce the motion of the device or component so that it remains proximate to a target site.

There have been many approaches that address fixation of medical devices. Some devices, such as a lead described in U.S. Pat. No. 4,269,198 to Stokes, employ fixed protrusions such as tines to engage body tissue. Other devices, such as the electrode assembly disclosed in U.S. Pat. No. 6,704,605 to Soltis et al., use a helical securing structure. U.S. Pat. No. 5,405,367 to Schulman et al. describes the use of barbs to hold a medical device such as a microstimulator in place.

Table 1 below lists documents that disclose some of the many devices and techniques pertaining fixation of medical devices. Some of the devices and techniques employ mechanical fixation structures such as tines or swellable membranes. Others employ adhesive properties to hold devices in place.

TABLE 1

| Patent Number | Inventors | Title |
|---|---|---|
| 6,240,321 | Janke et al. | Expandable seal for use with medical |
| | | device and system |
| 5,951,597 | Westlund et al. | Coronary sinus lead having expandable |
| | | matrix anchor |
| 5,545,206 | Carson | Low profile lead with automatic tine |
| | | activation |
| 5,405,367 | Schulman et al. | Structure and method of manufacture |
| | | of an implantable microstimulator |
| 4,768,523 | Cahalan et al. | Hydrogel adhesive |
| 4,658,835 | Pohndorf | Neural stimulating lead with fixation |
| | | canopy formation |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

In general, the invention is directed to fixation structures for medical devices implantable in a human or animal body, as well as medical devices incorporating such fixation structures. Such devices can include neurostimulators, sensors, electrodes, and the like. When the devices are implanted, it is generally desirable that migration of an implanted device be restricted. The invention presents easily implantable devices that help reduce migration.

Various embodiments of the present invention provide solutions to one or more problems existing in the prior art with respect to fixation mechanisms for implantable medical devices. These problems include the migration of medical devices from a desired implantation site. An additional problem is the reduced therapeutic efficacy that may result when a medical device migrates from its intended implantation site. Additional problems relate to the time and skill required in placement of conventional fixation mechanisms, such as sutures.

Various embodiments of the present invention are capable of solving at least one of the foregoing problems. In one exemplary embodiment, an implantable device includes a non-expandable element and one or more expandable elements. The expandable elements are constructed of a hydrogel material. During implantation, the expandable elements are in a dehydrated state, in which the expandable elements are smaller. But when implanted in the body of a patient, the expandable elements absorb water from the body tissues and assume a hydrated state. In the hydrated state, the expandable elements have a larger perimeter. Expansion of the expandable elements resists migration of the implanted device.

In comparison to known fixation mechanisms, various embodiments of the invention may provide one or more advantages. The invention can provide fixation for a variety of medical devices, including but not limited to self-contained stimulators and lead-mounted electrodes, without the need for sutures or other mechanisms requiring surgical placement. Rather, the fixation mechanism is generally self-deploying. In addition, the invention provides for a small profile during implantation, allowing implantation to be made by less invasive techniques.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a perspective view of an expandable device in a dehydrated state according to an embodiment of the invention.

FIG. 2 is a perspective view of the device of FIG. 1 in a hydrated state.

FIG. 3 is a cross section of an exemplary syringe that may be used to implant a device such as the device depicted in FIG. 1.

FIG. 4 is a cross section of a tissues that have received devices in a dehydrated state, according to two embodiments of the invention.

FIG. 5 a cross section of the tissues of FIG. 4, with the devices in hydrated states.

FIG. 6 is a perspective view of another expandable device in a dehydrated state according to an embodiment of the invention.

FIG. 7 is a perspective view of the device of FIG. 6 in a hydrated state.

FIG. 8 is a block diagram of an implantable stimulator according to an embodiment of the invention.

### DETAILED DESCRIPTION OF THE PREFERRED EMBODIMENTS

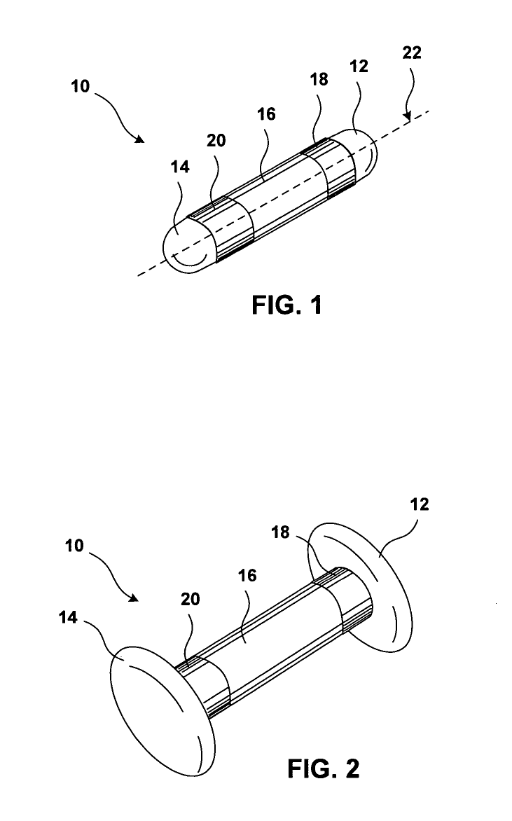

FIG. 1 depicts an exemplary medical device 10 configured to be implanted in a human or animal body. For purposes of illustration, FIG. 1 depicts a self-contained device such as a microstimulator or a sensor. The microstimulator may be a neurostimulator or muscle stimulator. The sensor may be configured to sense a variety of conditions, such as pressure, flow, temperature, fluid level, contractile force, pH, chemical concentration, or the like. Medical device 10 is self-contained in that it is not physically coupled to any other medical device by a lead or other connection. Medical device 10 can, for example, receive power from or wirelessly communicate with an external control device. In another embodiment, medical device 10 operates with an internal power supply. The invention is not limited to any particular medical device. Nor is the invention limited to self-contained medical devices, but encompasses medical devices that include leads or that are otherwise not self-contained.

Exemplary medical device 10 is shown in FIG. 1 in a first, miniature configuration. Exemplary medical device 10 is shown in FIG. 2 in a second, expanded configuration. In the first configuration, medical device 10 has a shape similar to that of a capsule or a grain of rice. In the second configuration, medical device 10 has a shape similar to that of a dumbbell. Medical device 10 includes two expandable elements 12 and 14, which are constructed of a biocompatible hydrogel material. Hydrogel materials that are believed to have wide applicability are the polyacrylonitrile copolymers as described in U.S. Pat. Nos. 4,943,618 and 5,252,692, which are incorporated herein by reference. By controlling relative amounts of copolymers, it is often possible to regulate physical qualities of the hydrogel such as flexibility and amount of expansion.

In general, hydrogels can assume a dehydrated state and a hydrated state. A hydrogel element in its dehydrated state is generally substantially smaller than the element in its hydrated state. A hydrogel element in its dehydrated state, when implanted in the body of a patient and placed in contact with body fluids, absorbs water and expands, assuming a hydrated state.

In this way, the capsule shaped device shown in FIG. 1 becomes the dumbbell shaped device shown in FIG. 2 when device 10 is implanted in the body of a patient and comes in contact with bodily fluids. Depending upon the composition of the hydrogel in expandable elements 12 and 14, and the amount of fluid, it may take expandable elements 12 and 14 from a few minutes to a few hours to expand.

Medical device 10 includes a non-expandable element 16 that may be constructed from any biocompatible material such as polyurethane or silicone. Non-expandable element 16 may take the form of a housing, which may be constructed from any of a variety of biocompatible materials such as silicone, polyurethane, titanium, stainless steel, fluoropolymer and hydrogel. In the embodiment shown in FIGS. 1 and 2, non-expandable element 16 is elongated and substantially cylindrical, and provides a platform for sensing or stimulating elements, depicted in FIGS. 1 and 2 as electrodes 18 and 20. However, medical device 10 may have any of a variety of shapes and sizes. In addition, non-expandable element 16 can serve as a housing for any electronic or other internal components of medical device 10. When medical device 10 is a self-contained stimulator, for example, non-expandable element 16 can house components such as a pulse generator, a wireless telemetry interface, a power supply and a processor that controls delivery of stimulations.

Expandable elements 12 and 14 can be coupled to non-expandable element 16 in any manner, such as by adhesive or by shaping expandable elements 12 and 14 to lock with non-expandable element 16. As shown in FIGS. 1 and 2, expandable elements 12 and 14 can be deployed on the ends of non-expandable element 16, but the invention encompasses embodiments in which the expandable elements are deployed elsewhere.

When expandable elements 12 and 14 are in the dehydrated state, and device 10 is in a miniature configuration, the dimensions of device 10 can be selected such that device 10 can fit inside the bore of an insertion device, such as needle, hollow trocar, endoscope, catheter or cannula. In particular, device 10 can fit through an sleeve oriented substantially parallel to long axis 22. Dimensions of implant 10 in the dehydrated state can be approximately one to seven millimeters in diameter (transverse to axis 22) and approximately ten to twenty millimeters in length (parallel to axis 22). The invention encompasses other shapes and dimensions as well. The dimensions of medical device 10 can depend upon the internal components of medical device 10.

The invention encompasses various shapes and dimensions of expandable elements 12 and 14. As shown in FIGS. 1 and 2, the diameter of expandable elements 12 and 14 in the dehydrated state can be comparable to the diameter of non-expandable element 16. While in the expanded state, by contrast, the diameter of expandable elements 12 and can be at least approximately two times, and more preferably at least approximately three times the diameter of non-expandable element 16. Expandable elements 12 and 14 swell radially. That is, expandable elements 12 and 14, expand outward from axis 22. While in the dehydrated state, expandable elements 12 and 14 have a small perimeter, but in the hydrated state, expandable elements 12 and 14 have a larger perimeter. As noted above, the degree of expansion can be regulated. The hydrogel can be configured, for example, to expand approximately two to five times when hydrating.

FIGS. 1 and 2 show a medical device having one non-expandable element and two expandable elements. As discussed below, the invention encompasses embodiments that include multiple non-expandable elements and multiple expandable elements. As depicted in FIGS. 1 and 2, medical device 10 is not directionally specific, and would function the same way when turned end for end. The invention encompasses embodiments, however, in which the medical device is directionally specific. The invention encompasses, for example, a medical device that has a larger expandable element on its proximal end and a smaller expandable element on its distal end.

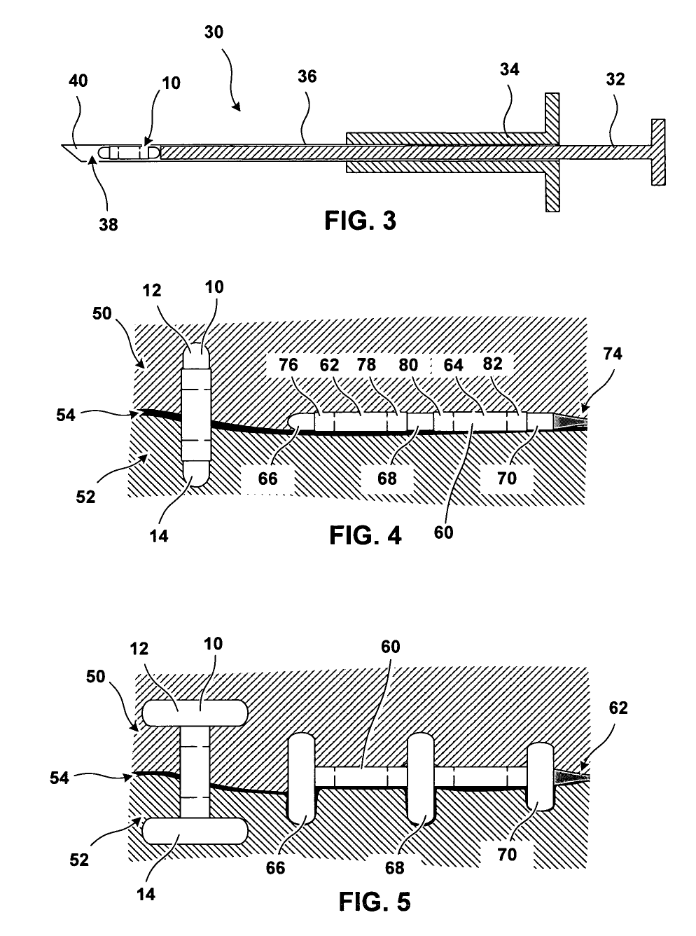

FIG. 3 is a cross-sectional diagram of a device 30 that can implant a medical device such as medical device 10 in a patient. Device 30 comprises a syringe, which includes a plunger member 32, a body member 34 and a hollow needle 36 having a lumen 38. Needle 36 is fixedly coupled to body member 34, while plunger member 32 is free to move in lumen 38. Lumen 38 of needle 36 has been enlarged to show medical device 10, in a miniature configuration with hydrogel elements in the dehydrated state, disposed in lumen 38. As depicted in FIG. 2, medical device 10 in the miniaturized configuration is an elongated, substantially rice-shaped device, and sized to fit inside lumen 38.

Distal end 40 of needle 36 includes a sharp point that can pierce tissue such as the skin, the mucosa of the gastrointestinal tract, a body organ or a tissue mass. Distal end 40 further includes an opening through which medical device 10 may be expelled from lumen 38 by depressing plunger member 32 with respect to body member 34.

Device 30 is not the only device that can be used to implant a medical device such as medical device 10. For example, a physician can implant medical device 10 by making an incision in the skin, introducing an insertion device such as a catheter into the body of the patient, guiding the insertion device to a target site, pushing medical device 10 through the insertion device, and withdrawing the insertion device.

In general, implantation of a medical device in a miniature configuration is less invasive than a surgical procedure to implant a the medical device in its enlarged configuration. The medical device can be delivered to a target site in a miniature configuration, and expand on its own to its enlarged configuration. In some cases, a fluid can be injected into the implantation site to accelerate expansion.

FIGS. 4 and 5 are cross-sections of neighboring tissue layers 50 and 52. As depicted in FIGS. 4 and 5, tissue layers 50 and 52 are separated by a boundary 54, such as bone or fascia. FIGS. 4 and 5 depict deployment of medical device 10 from FIGS. 1 and 2. Medical device 10 is shown deployed with expandable element 12 in tissue layer 50, and expandable element 14 in tissue layer 52. Boundary 54 can be less resilient than tissue layers 50 and 52, and can prevent passage of expandable elements 12 and 14 in their enlarged, hydrated states, thereby reducing migration of medical device 10. For example, when boundary 54 represents the sacrum, medical device 10 can be deployed through a sacral foramen, and expandable elements 12 and 14 can prevent medical device 10 from migrating from the foramen. Deployed in such as fashion, electrodes 18 and 20 deployed on medical device 10 can be configured to sense or stimulate sacral nerves, e.g., for neurostimluation therapy for sexual dysfunction or urinary or fecal incontinence.

FIGS. 4 and 5 also depict deployment of medical device 60, which is another embodiment of the invention. Medical device 60 is similar to medical device 10 in some respects. Medical device 10 includes one non-expandable element 16 and two expandable elements 12 and 14, and medical device 60 includes two non-expandable elements 62 and 64, with three expandable elements 66, 68 and 70. Like medical device 10, medical device 60 is substantially cylindrical. As shown in FIGS. 4 and 5, however, medical device 60 defines a smaller diameter than medical device 10. Medical device 60 can be deployed through a catheter, a cannula or other medical instruments.

As shown in FIG. 5, expandable elements 66, 68 and 70 have expanded to a hydrated state when placed in contact with the body fluids present in the tissues. Expandable elements 66, 68 and 70 may be, but need not be, the same size as one another when in the hydrated state. As shown in FIG. 5, for example, expandable element 70, when in the hydrated state, is smaller than expandable elements 66, and 68. Expandable elements 68 and 70 can include central apertures to permit passage of electrical components such as lead wires.

The materials used to make medical device 60 may be the same as the materials used to make medical device 10. In some embodiments of the invention, first non-expandable element 62 may be constructed of a first material, such as polyurethane, and second non-expandable element 64 may be constructed from a second material, such as silicone

Medical device 60 includes a lead 74. Lead 74 can be coupled to non-expandable element 64 through a central aperture in expandable element 70. Lead 74 includes one or more conductors that are electrically coupled to electrodes 76, 78, 80 and 82. The conductors can further be electrically coupled to an implantable medical device (not shown). One example of such an implantable medical device is an implantable pulse generator, which generates stimulating pulses. The stimulating pulses can be delivered to tissue via electrodes 76, 78, 80 and 82. Another example of such an implantable medical device is a sensor that senses electrical parameters, temperature, or other physiological aspects.

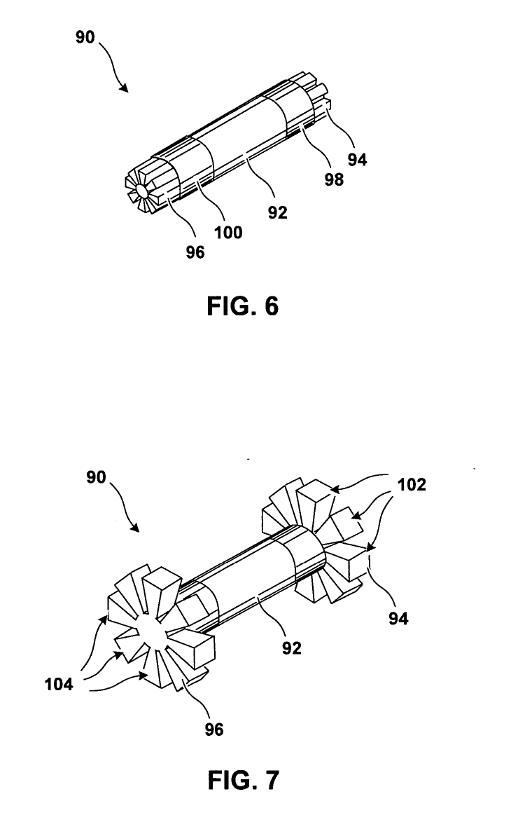

FIGS. 6 and 7 depict another embodiment of an exemplary medical device 90 configured to be implanted in a human or animal body. Medical device 90 is similar to medical device 10 shown in FIGS. 1 and 2, in that medical device 90 includes an elongated non-expandable element 92 and two expandable elements 94 and 96. In the embodiment shown in FIGS. 6 and 7, medical device 90 also includes stimulating elements such as electrodes 98 and 100. The materials used to make medical device 10 could also be used to make medical device 90.

Exemplary medical device 90 is shown in FIG. 6 in a miniature configuration, and is shown in FIG. 7 in an expanded configuration. In the miniature configuration, medical device 90 has a shape and dimensions comparable to that of a medical device 10 shown in FIG. 1. When implanted in a body of a patient, expandable elements 94 and 96 swell radially when placed in contact with bodily fluids, and device 90 assumes an expanded configuration. In the expanded configuration, expandable elements 94 and 96 have a larger perimeter, giving medical device 90 a modified dumbbell shape. Expandable elements 94 and 96 include a plurality of projections 102, 104 separated by gaps.

Expandable elements 94 and 96 can be prepared in a number of ways. One technique is to shape expandable elements 94 and 96 when expandable elements 94 and 96 are in the expanded hydrated state, then desiccate expandable elements 94 and 96 to put them in a dehydrated state and thereby reduce their size. Molding and cutting are two exemplary techniques for shaping expandable elements 94 and 96.

Multiple projections may offer one or more advantages. For example, gaps between projections 102, 104 can facilitate flow of fluid in some deployments. In other deployments, cells such as fibroblasts and fibrous tissue can occupy the gaps, thereby anchoring expandable elements 94 and 96 more firmly.

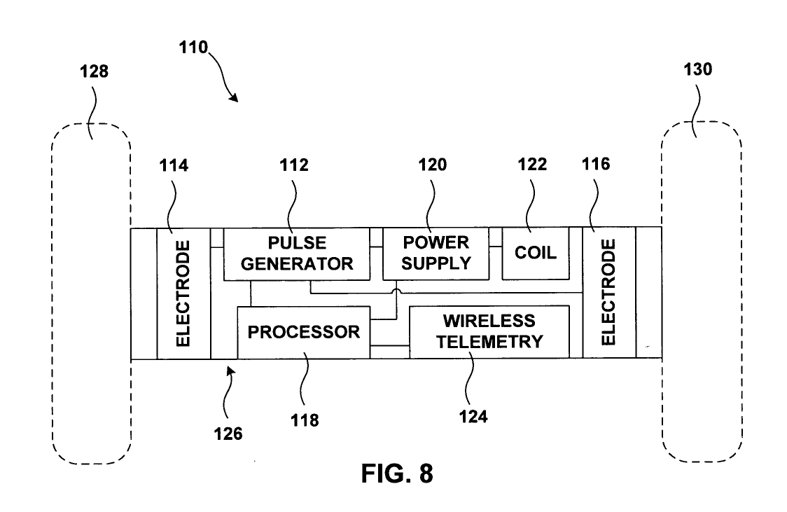

FIG. 8 is a block diagram of an exemplary medical device 110 that can employ many of the features described above. Medical device 110 is an example of a self-contained implantable stimulator. Stimulator 110 includes pulse generator 112, which supplies electrical stimulations to the target tissue via electrodes 114, 116, which are exposed to the tissue. Pulse generator 112 supplies stimulations under the direction of processor 118. Processor 118 may comprise a microprocessor, application specific integrated circuit, a programmable logic chip, or other controlling circuitry.

A power supply 120, such as a capacitor or a battery, supplies power to pulse generator 112 and processor 118. In the embodiment depicted in FIG. 8, medical device 110 includes an inductive coil 122 configured to receive energy from an external energy source. Medical device 110 also includes a wireless telemetry interface 124 configured to wirelessly transmit or receive data or instructions. Pulse generator 112, processor 118, power supply 120, coil 122 and wireless telemetry interface 124 are housed inside non-expandable element 126.

Expandable elements 128, 130 expand from a dehydrated state to a hydrated state when medical device 110 is implanted in the body of a patient. Expansion of expandable elements 128, 130 restricts migration of medical device 110. As a result, electrodes 114, 116 tend to remain proximate to the target tissue.

Medical device 110 can include components other than or in addition to the components depicted in FIG. 8. For example, medical device 110 can incorporate an accelerometer or a sensor, such as a sensor configured to sense conditions such as pressure, flow, temperature, fluid level, contractile force, pH or chemical concentration. The invention is not limited to the specific embodiment depicted in FIG. 8.

In addition, FIG. 8 depicts electronic components such as electrodes as housed inside non-expandable element 126. The invention also supports embodiments in which one or more components are deployed on or in expandable elements 128, 130. For example, an electrode can be deployed on the surface of an expandable element such that the electrode moves away from non-expandable element 126 when the expandable element expands.

The preceding specific embodiments are illustrative of the practice of the invention. It is to be understood, therefore, that other expedients known to those skilled in the art or disclosed herein may be employed without departing from the invention or the scope of the appended claims. For example, the invention is not limited to an implant having the shape and illustrative dimensions described above.

Furthermore, the invention is not limited to the particular shapes of expandable elements depicted in the figures. As shown in FIGS. 1, 2, 6 and 7, the expandable elements have substantially circular perimeters, and the perimeters expand as the expandable elements assume the hydrated state. In some embodiments, the perimeters of the expandable elements can by substantially elliptical or triangular, for example. In addition, the invention encompasses embodiments in which the expandable elements expand further in one direction than in another. The invention also encompasses embodiments in which one or more expandable element is folded or rolled to reduce its profile in the dehydrated state. As such an expandable element expands, the expandable element automatically unfolds or unrolls.

Although the invention is described as useful in application with the neurostimulation, the invention is not limited to that application. Furthermore, the invention can be deployed via implantation techniques in addition to those described above. The invention further includes within its scope methods of making and using the implants described above.

In the appended claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

1. An implantable medical device comprising:
a non-expandable element; and
a hydrogel element coupled to the non-expandable element;
wherein the hydrogel element has a first perimeter in a dehydrated state and a second perimeter in a hydrated state, and
wherein the hydrogel element is configured to expand from the first perimeter to the second perimeter when the hydrogel element is in contact with a body fluid of a patient.

2. The device of claim 1, wherein the hydrogel element is a first hydrogel element, the device further comprising a second hydrogel element coupled to the non-expandable element and configured to expand from a dehydrated state to a hydrated state.

3. The device of claim 2, wherein the non-expandable element has a proximal end and a distal end, wherein the first hydrogel element is coupled to the proximal end, and the second hydrogel element is coupled to the distal end.

4. The device of claim 1, wherein the non-expandable element is constructed of at least one of polyurethane, silicone, titanium, stainless steel, fluoropolymer or hydrogel.

5. The device of claim 1, wherein the non-expandable element is substantially cylindrical and has a diameter from approximately one millimeter to approximately seven millimeters.

6. The device of claim 1, wherein the length of the device is from ten millimeters to twenty millimeters.

7. The device of claim 1, wherein the second perimeter is approximately two times to five times larger than the first perimeter.

8. The device of claim 1, further comprising an electrode coupled to the non-expandable element.

9. The device of claim 8, further comprising a lead comprising a conductor electrically coupled to the electrode.

10. (canceled)

11. The device of claim 8, further comprising a pulse generator coupled to the electrode, wherein the pulse generator is housed inside the non-expandable element.

12. (canceled)

13. The device of claim 1, wherein the non-expandable element is a first non-expandable element and wherein the hydrogel element is a first hydrogel element, the device further comprising:
a second non-expandable element coupled to the first hydrogel element; and
a second hydrogel element coupled to the second non-expandable element.

14. The device of claim 1, further comprising a sensor coupled to the non-expandable element, wherein the sensor is configured to sense at least one of pressure, flow, temperature, fluid level, contractile force, pH or chemical concentration.

15. An implantable electrical stimulation device comprising:
a non-expandable element housing an implantable pulse generator; and
a hydrogel element coupled to the non-expandable element;
wherein the hydrogel element is configured to expand from a dehydrated state to a hydrated state.

16. The device of claim 15, further comprising a processor configured to control the pulse generator, wherein the non-expandable element houses the processor.

17. The device of claim 15, further comprising a lead comprising a conductor electrically coupled to the pulse generator.

18. (canceled)

19. The device of claim 15, wherein the non-expandable element is substantially cylindrical and has a diameter from approximately one millimeter to approximately seven millimeters.

20. The device of claim 15, wherein the length of the device is from ten millimeters to twenty millimeters.

21. The device of claim 15, wherein the hydrogel element is configured to expand in dimension approximately two times to five times when expanding from the dehydrated state to the hydrated state.

22-27. (canceled)

28. An implantable medical device comprising:
non-expandable element means; and
expandable element means coupled to the non-expandable element means;
wherein the expandable element means is configured to expand from a first perimeter to a second perimeter when the expandable element means is in contact with a body fluid of a patient.

29. The device of claim 28, further comprising an electrode means coupled to the non-expandable element means, and a stimulating means coupled to the electrode means.

30. (canceled)

31. The device of claim 28, wherein the device is a substantially rice-shaped device prior to expansion of the expandable element means from the first perimeter to the second perimeter.

32. The device of claim 28, wherein the device is a substantially dumbbell-shaped device after the expansion of the expandable element means from the first perimeter to the second perimeter.

33. The device of claim 28, wherein the non-expandable element means is substantially cylindrical and has a diameter from approximately one millimeter to approximately seven millimeters.

34. The device of claim 28, wherein the length of the device is from ten millimeters to twenty millimeters.

35. The device of claim 28, wherein the expandable element means is configured such that the second perimeter is approximately two times to five times larger than the first perimeter.

36. A method for implanting a medical device, comprising:
placing a medical device into a bore of an insertion device, the medical device including a non-expandable element and a hydrogel element coupled to the non-expandable element, the hydrogel element being in a dehydrated state;
inserting the insertion device into the body of a patient; and
ejecting the medical device from the insertion device proximate to a target tissue,
wherein the hydrogel element is configured to expand from the first perimeter to the second perimeter when the hydrogel element is ejected from the insertion device.

37. The method of claim 36, wherein the medical device comprises an implantable neurostimulator or an implantable physiological sensor.

38. (canceled)

39. The method of claim 36, wherein the insertion device comprises one of a needle, a hollow trocar, an endoscope, a catheter or a cannula.

40. An implantable electrical stimulation device comprising:
a non-expandable element housing a sensor; and
a hydrogel element coupled to the non-expandable element;
wherein the hydrogel element is configured to expand from a dehydrated state to a hydrated state.

41. The device of claim 40, wherein the sensor is configured to sense at least one of pressure, flow, temperature, fluid level, contractile force, pH or chemical concentration.

42. (canceled)

43. The device of claim 40, wherein the non-expandable element is substantially cylindrical and has a diameter from approximately one millimeter to approximately seven millimeters.

44. The device of claim 40, wherein the length of the device is from ten millimeters to twenty millimeters.

45. The device of claim 40, wherein the hydrogel element is configured to expand in dimension approximately two times to five times when expanding from the dehydrated state to the hydrated state.
